(19) United States
(12) Patent Application Publication
(10) Pub. No.: US 2008/0140184 A1
(43) Pub. Date: Jun. 12, 2008

(54) Scaffold device

(76) Inventor: J. Lee Pellegrino-Gensey, Ewing, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/955,100

(22) Filed: Sep. 30, 2004

(51) Int. Cl. A61F 2/02; A61K 38/28

(52) U.S. Cl. 623/1.34; 623/23.76; 623/1.42; 514/3

(57) ABSTRACT

A porous device to create a vascularized transplant site within a patient that improves the survivability of therapeutic cells contained therein. The device comprises a radiopaque material that allows its detection by radiographic techniques so as to allow implantation of therapeutic tissues after the device has been implanted.

### FIELD OF THE INVENTION

The field of this invention is implantable organs, tissues, organoids, or transplanted cells formed on, within, or about biocompatible artificial matrices.

### BACKGROUND OF THE INVENTION

Devices have been suggested for transplanting tissue such as, e.g., cells, organoids, organs or tissue to create artificial organs when the patient's own organ function is lost due to disease or injury. Such devices may also be used to transplant cells to specific sites to repair tissue damaged due to infection or injury.

Such devices, commonly called scaffolds, are typically seeded with tissue that express a necessary substance, such as, e.g., insulin, and implanted in areas where such substances will be transported to other areas of the body. Quite often, though, enhanced survivability requires a highly vascularized site to bring in necessary nutrients and oxygen and to remove wastes. When the scaffolds are first implanted, even in highly vascularized sites, the vasculature itself does not extend into the scaffold. Thus, tissue survivability is always a concern with such implants.

One solution to this problem is to implant the scaffold into a surgical site before implantation to allow vasculature to grow into the device. Afterward, the scaffold can be implanted with the desired tissue. The problem with this solution, however, is that, in order to seed a scaffold with tissue after the scaffold has been implanted requires that the surgeon have the ability to locate the scaffold in the patient. The present invention addresses this problem.

### SUMMARY OF THE INVENTION

The current invention provides for a device for the transplantation of tissues such as, e.g., cells, organoids, or organs for the effective treatment of a disease or injury. The device contains radiopaque material to allow one to locate the device using standard scanning devices.

The current invention additionally calls for an insert defining a lumen with specified dimensional restrictions that can be filled with therapeutically effective cells, tissues, organoids, or organs after removal of the insert.

The current invention further calls for a device seeded with tissues that provide a therapeutic benefit.

The current invention still further calls for a device that has sufficient porosity to allow for adequate in-growth of vasculature.

The current invention still further calls for a device that is treated to reduce inflammation, to reduce fibrosis and/or to enhance angiogenesis.

The current invention also comprises a method for transplanting a scaffold device, allows sufficient time for vasculature to grow into the device, and is seeded with cells.

The current invention also comprises a method for transplanting a scaffold device, allow sufficient time for vasculature to grow into the device, allow time for the device to biodegrade causing a cavity in the vasculature to result, and seed the cavity with cells.

These embodiments and more are described more fully in the following sections.

### BRIEF DESCRIPTION OF THE DRAWINGS

FIG. 1 shows the device of the present invention in cross section.

FIG. 2 shows the device of the present invention in plan view.

FIG. 3 shows the device of the present invention in side view. The device (10) contains a central insert (40), which has a height (D) of not more than 1 mm and is formed to fit exactly in the central lumen, and protrudes from the wall of the device a distance E.

FIG. 4 shows the plug that is used to seal device of the present invention.

FIG. 5 shows the removable insert containing the radiopaque marker that is inserted into the lumen of the device of the present invention.

### DETAILED DESCRIPTION OF THE INVENTION

### Definitions

The term “scaffold” as used herein refers to a three-dimensional device that is suitable for containing cells for implantation.

The term “biocompatible” is meant that the device of the present invention does not substantially adversely affect any desired characteristics of the cells to be seeded within the device, or the cells or tissues in the area of a mammalian subject where the device is to be implanted, or any other areas of the mammalian subject.

The term “biodegradable” or “absorbable” is meant that the device will be gradually degraded or absorbed and its byproducts are safely broken down or cleared after the device is delivered to a site of interest inside the body of a mammalian subject.

The term “implantable” as used herein refers to a biocompatible three-dimensional scaffold that is inserted within a mammalian subject.

The term “a mammalian subject” is meant to include a primate, porcine, canine, murine, and human subject, and particularly a human subject.

The term “therapeutic cell” is meant as any cell, tissue, organoid or combinations of cells that provides a structural function or a therapeutic response expressing or secreting a therapeutic factor such as a protein, cytokine, hormone or growth factor. The therapeutic cells may be autologous, allogeneic, or xenogeneic cells. The cells can be stem cells or primary or expanded cells that are undifferentiated or partially to fully differentiated and/or genetically engineered cells.

The term “pharmaceutical agent that promotes survival of mammalian cells” is meant to include growth factors, extracellular matrix proteins, and biologically relevant peptide fragments, that promote survival of mammalian cells seeded in the device by facilitating cell attachment, promoting cell proliferation, inhibiting apoptosis, or blocking or inhibiting the cytotoxic functions of immune cells of the recipient towards the mammalian cells being implanted via the matrices. Such pharmaceutical agents include, but are not limited to, members of TGF-b family, including TGF-b1, 2, and 3, bone morphogenic proteins (BMP-2, -3, -4, 5 6, -11, -12, and -13), fibroblast growth factors-1 and -2, platelet-derived growth factor-AA, and -BB, platelet rich plasma, insulin growth factor (IGF-I, II) growth differentiation factor (GDF-5, -6, -8, -10, -15) vascular endothelial cell-derived growth factor (VEGF), pleiotrophin, endothelin, nicotinamide, hypoxia inducible factor 1-alpha, glucagon like peptide-I (GLP-1) and II, Exendin-4, retinoic acid, parathyroid hormone, tenascin-C, tropoelastin, thrombin-derived peptides, cathelicidins, defensins, laminin, biological peptides containing cell- and heparin-binding domains of adhesive extracellular matrix proteins such as fibronectin and vitronectin, MAPK inhibitors, and combinations thereof. Pharmaceutical agents can also include small molecules that up-regulate the production or activity of endogenous grow factors, anti-rejection agents, analgesics, anti-oxidants, anti-apoptotic, anti-inflammatory agents, angiogenic agents and combinations thereof.

### The Device

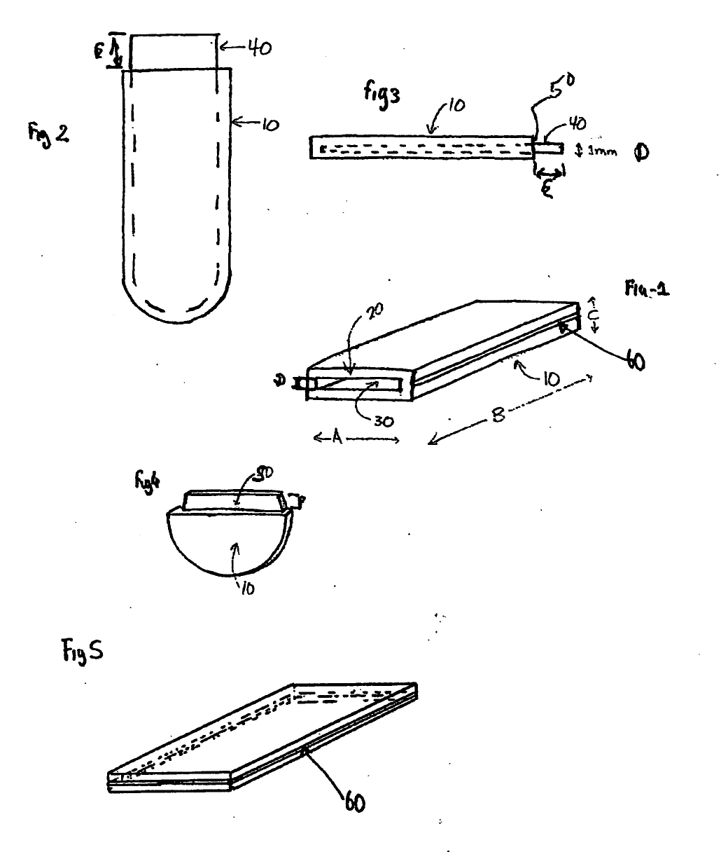

A view of the device is shown in FIG. 1. The current invention consists of a device (10) here shown with a width (A), length (B) and depth (C). The walls of device (20) surround a central lumen (30), which is limited to a height (D). The device (10) contains an insert (40), which is formed to fit in the central lumen (30), and protrudes from the end (50) of the device (10) a distance (E). A radiopaque material is in, on, or otherwise an integral part of the device (10).

Within the device are placed tissues, such as, e.g., cells, organoids, and/or organs to mimic or replace the function of an organ specific to the cellular material placed therein.

In a preferred embodiment, the device (10) is made from a biocompatible polymer. Such biocompatible polymers may be either biodegradable or non-biodegradable. However, biodegradable polymers are more preferred. More preferable is a device (10) made up exclusively of biodegradable materials.

The device (10) may be treated with factors to reduce inflammation, fibrosis and/or to enhance angiogenesis through the pores of the device walls (20). Such materials are known to those skilled in the art.

In a preferred embodiment, the radiopaque marker is incorporated into the biocompatible polymer that comprises the walls (20) of the device.

The purpose of the insert (40) is to fit into and even fill up the lumen (30) and to act as an impermeable structure to prevent the complete in-growth of the tissue into the lumen (30), assuming it is implanted prior to seeding, so as to allow seeding with tissues at an appropriate time. It is preferred that a tight fit of the insert (40) in to the lumen (30) be achieved in order to avoid premature removal of the insert (40). It is also preferred that the insert (40) protrude from the lumen (30) so as to be more easily gripped when the time comes to remove it.

The insert (40) may also serve to give structural stability or strength to the device (10). Preferably, the insert (40) is manufactured from a material that does not permit the attachment of cells or tissues. It may be made of any biocompatible material. Such materials are known to those skilled in the art. In situations where the device (10) will be implanted prior to seeding, the device (10) with the insert (40) placed in the lumen (30) is implanted. The insert (40) prevents growth of vasculature into all or part of the lumen (30). Following a time sufficient to induce a high amount of vascular in-growth towards the lumen (30), the insert (40) will be removed and the therapeutic cells introduced. Thereafter, the insert (40) may be either replaced or discarded.

The device (10) is shown as rectangular. However, it may be formed in any manner that forms an interior space or cavity (30) that could be adapted for the placement and containment of therapeutic tissue. Such other shapes could be, for example, cylindrical, spherical, or other frusta-conical shapes.

However it is formed, it is preferred that the dimensions of the cavity (30) not exceed in any one of the dimensions twice the distance of optimal diffusion of nutrients and oxygen. Thus, for any type of tissue that is placed in the lumen (30), none of it would be greater than the distance of optimal diffusion. In the case of tissue that is capable of producing insulin, and in the case of a device (10) that is rectangular in cross section, it is preferred that the height (D), being the smallest dimension, not exceed about 2 mm. More preferably, the height (D) should not exceed about 1 mm. More preferably yet, the height (D) should be from about is 0.5 mm to 1 mm.

The device (10) may have ports for injection of cells, or it may have a permanent opening that can be closed by the insert (40).

The device (10) should be constructed in such a manner to maximize the surface area of the device (10) thereby allowing for the greatest area for new blood vessel infiltration and maintain a minimal height (D).

The size of the lumen (30) is dependent on the number and type of therapeutic cells to be implanted. Such factors should be easily determinable by those skilled in the art with minimal experimentation.

The wall (20) of the device (10) must be suitable to encourage vascularization. A preferred structure for promoting in-growth of vasculature is one where the wall (20) is porous, and the pores are open and sufficiently sized to permit diffusion of nutrients and waste materials while maintaining a high degree of therapeutic cell retention.

The pores of the device (10) can be achieved by a number of different methods known to those skilled in the art, including punching, drilling, particle leaching, foaming, weaving, knitting, or lyophilization. It is preferred that the pore size of the wall (20) of device (10) be in the range of 10-1000 microns, preferably 10-500 microns. More preferably it is 10-100 microns.

Ideally, the device wall (20) should be thin, preferably less than 200 microns. Any method that produces such a wall thickness, such as, for example, knitting, should be sufficient to allow rapid cellular in-growth.

One of ordinary skill in the art will appreciate that the selection of a suitable material for forming the said device (10) depends on several factors. The more relevant factors in the selection of the appropriate material include bioabsorption (or biodegradation) kinetics; in vivo mechanical performance; cell response to the material in terms of cell attachment, proliferation, migration and differentiation; and biocompatibility. Other relevant factors, which to some extent dictate the in vitro and in vivo behavior of the material, include the chemical composition, spatial distribution of the constituents, the molecular weight, the degree of crystallinity, and monomer content in the case of polymeric materials. The surface properties of the materials can also be optimized to achieve the desired hydrophilicity. The methods that are used to construct the polymers used in the device of the present invention are disclosed in US patent application US20040062753 A1 and U.S. Pat. No. 4,557,264 issued Dec. 10, 1985 assigned to Ethicon, Inc, which are hereby incorporated by reference.

Additional scaffolds that can be employed in the device of the present invention are disclosed in U.S. provisional patent applications 60/483,804; and 60/510,507; and U.S. patent application Ser. No. 10/405,693 which are hereby incorporated by reference.

### Implantation of the Device

The device (10) is to be implanted in a mammal, preferably a human being. The device (10) may be pre-implanted in the body to pre-condition the implantation site before cells are introduced into the device (10). That is, the device (10) is implanted without therapeutic tissue, in order to allow the necessary vasculature to grow into the device (10). Such vasculature will ultimately supply the necessary nutrients and oxygen to such tissues when they are subsequently placed in the device (10).

The cavity (30) of the device (10) is preserved during this pre-implantation period by an insert (40). This insert (40) also serves to prevent the collapse of the device (10). After implantation, vasculature is allowed to grow into the device (10), but not completely fill the cavity (30). When sufficient vasculature has grown, the insert (40) is removed, and the device (10) is seeded with therapeutic tissue. After seeding, the insert (40) may or may not be replaced, depending on the need to hold the therapeutic tissue in place.

Alternatively, the device (10) may be seeded with therapeutic tissue prior to implantation. In this case, the insert (40) acts more as a plug to keep the therapeutic tissue in place until vasculature grows into the device (10).

The device (10) may be combined with biologically active compounds or factors to enhance the performance of the device (10). Such compounds or factors may enhance the growth of vasculature, or have anti-inflammatory or angiogenic properties, or other attributes that would enhance cell survival. Such compounds or biologically active factors may also enhance the proliferation and/or differentiation of the therapeutic cells.

The device (10) may be implanted subcutaneously, or in a body cavity or in or around a tissue or organ. Such sites include but are not limited to the omentum, mesentery, peritoneal tissue, or intestinal subserosal/mucosal space.

### Radiopaque Marker

The purpose of the radiopaque marker is to allow the detection of the device (10) after it is implanted in the body of the mammal. It can be placed on or in the walls (20) of the device (10) itself or on or in the insert (40). It may also be placed on or in both the walls (20) and the insert (40). Preferably, it is placed in such a way that the outline of the device (10) is easily recognizable to a physician who is using a traditional scanning device such as, e.g., an x-ray, CAT scanner, and/or NMR.

The marker is can be any material that is radiopaque. That is, the material is anything that offers sufficient contrast, as compared to the device (10), when view by such scanning devices. Preferably, the placement of such markers will allow the physician to be able to determine both the location and orientation of the device (10) when it is in the body of the mammal. Such knowledge will allow the physician the ability to place therapeutic tissue into the device (10) using low-invasive surgical techniques that will augment the healing process.

In the figures, a wire (60) is shown that runs the through the edges of the insert (40), or the walls (20) of the device (10). Other types of radiopaque materials include that which is a material possessing radiographic density higher than surrounding host tissue and has sufficient thickness to affect the transmission of x-rays to produce contrast in the image. Various devices having radiopacity are known in the art such as shown in U.S. Pat. Nos. 4,447,239; 5,354,257; and 5,423,849, herein incorporated by reference. A variety of radiopaque materials can be used for this purpose, such as barium sulfate and bismuth trioxide.

Other metals such as stainless steel, super-alloys, nitinol, and titanium having lower radiographic densities may also be used. Other elements that also could be used as radiopaque markers further include zirconium, barium, bismuth, and iodine. Preferably, the radio-opaque marker consists of a metal wire incorporated into the edge of the removable core. In another embodiment, the insert can contain radiopaque material at the center of a hollow shell of material.

It is not necessary for large amounts of radiopaque material to be incorporated into the device (10). Rather, it is sufficient for only that amount necessary to locate by traditional radiographic means.

### Therapeutic Tissue

Therapeutic tissue is defined as any cell, organs, organoids or combinations of cells that provides a structural function or a therapeutic response expressing or secreting a therapeutic factor such as a protein, cytokine, hormone or growth factor. The therapeutic cells may be autologous, allogeneic, or xenogeneic cells. The cells can be stem cells or primary or expanded cells that are undifferentiated or partially to fully differentiated and/or genetically engineered cells. Examples of such cells that could be used to treat diabetes include islets or any insulin-secreting cell, pancreatic stem cells, precursor or progenitor cells, pancreatic ductal cells, or genetically engineered insulin producing cells. Other disease states utilizing a variety of cell therapies could also benefit from the current invention including but are not limited to hepatocytes for the treatment of liver failure, chromaffin cells for chronic pain, cells that produce clotting factors for hemophilia, and cells that produce nerve growth factors for the treatment of neurodegenerative disease such as Parkinson's or Alzheimer's disease.

Other cells that can be therapeutically effective for different applications include bone marrow cells, umbilical cord blood cells, angioblasts, endothelial cells, osteoblasts, smooth muscle cells, kidney cells, stem cells, cardiovascular cells, myofibroblasts, fibroblasts, neural cells, and neural precursors.

### Use of Device for Therapy

The present invention can be used to localize transplanted cells, tissues, organoids, or organs that can provide a therapeutic benefit. Cells can either be derived directly from organs or tissues or can be cultured or genetically modified. Cell therapy has a broad application for treating major disease such as diabetes, as well as a wide range of other disorders. Having a vascularized environment for therapeutic cells to flourish, one could apply this intention to hepatocytes for the treatment of liver failure, chromaffin cells for chronic pain, cells that produce clotting factors for hemophilia, and cells that produce nerve growth factors for neurodegenerative disease such as Parkinson's or Alzheimer's disease.

What is claimed is:

1. An implantable, biocompatible scaffold device comprising a cavity-encasing wall surrounding a cavity and a radiopaque material.

2. The device of claim 1, further comprising a cavity insert.

3. The device of claim 1, wherein the radiopaque material comprises a wire.

4. The device of claim 4, wherein the wire is placed near the periphery of the device.

5. The device of claim 2, wherein the radiopaque material comprises a wire.

6. The device of claim 5, wherein the wire is also placed in the insert.

7. The device of claim 1, wherein the radiopaque material is selected from the group consisting of barium sulfate, bismuth trioxide, barium, bismuth, iodine, steel, steel alloys, nitinol, and titanium.

8. The device of claim 1, wherein the therapeutic tissue produces insulin.

9. A method of treating a therapeutic condition comprising implanting a scaffold device into a mammal, allowing sufficient time for vasculature to grow into the device, and implanting therapeutic tissue into the device, wherein the scaffold device comprises a cavity-encasing wall surrounding a cavity and a radiopaque material and wherein the therapeutic tissue is implanted into the cavity.

10. The method of claim 9, wherein after implanting the device into the mammal, a period of time greater than one day elapses before the therapeutic tissue is implanted.

11. The method of claim 8, wherein the radiopaque material comprises a wire.

12. The method of claim 8, wherein a physician uses a scanning device to locate the radiopaque material to locate the device prior to implanting the therapeutic tissue.

13. The method of claim 8, wherein the radiopaque material is selected from the group consisting of barium sulfate, bismuth trioxide, barium, bismuth, iodine, steel, steel alloys, nitinol, and titanium.

14. The method of claim 8, wherein the therapeutic tissue produces insulin.

15. A method of treating a therapeutic condition comprising implanting therapeutic tissue into a scaffold device and implanting the device into a mammal, wherein the scaffold device comprises a cavity-encasing wall surrounding a cavity and a radiopaque material and wherein the therapeutic tissue is implanted into the cavity.

16. The method of claim 15, wherein the therapeutic tissue produces insulin.
